(19) United States
(12) Patent Application Publication
(10) Pub. No.: US 2026/0108269 A1
(43) Pub. Date: Apr. 23, 2026

(54) MIDPALATAL PIEZOCORTICOMY GUIDE AND METHOD OF USING SAME

(71) Applicant: Svitlana KOVAL, Boca Raton, FL (US)

(72) Inventor: Svitlana KOVAL, Boca Raton, FL (US)

(21) Appl. No.: 18/919,416

(22) Filed: Oct. 17, 2024

(51) Int. Cl. A61B 17/3209; A61B 17/32; A61C 7/10

(52) U.S. Cl. CPC A61B 17/3209; A61C 7/10; A61B 2017/320052

(57) ABSTRACT

An exemplary maxillary incision guide for a maxilla having a palatial region within a palatial perimeter may comprise a retainer for retaining the guide to the maxilla at a point outside the palatial perimeter, a base suspended from the retainer by a plurality of posts extending from the retainer across the palatial perimeter. An exemplary base of an exemplary maxillary incision guide may be configured to be in contact with a portion of the palatial region and provide access to the same through a plurality of slots formed through the thickness of the base.

### FIELD OF THE INVENTION

Disclosed are embodiments of the invention that relate to, among other things, devices and methods for guiding ultrasound bone separating cuts in the human mouth, in particular, for use with maxillary expansion techniques and devices.

### BACKGROUND

Mini-screw-Assisted Rapid Palatal Expansion (MARPE) is used in patients with posterior cross-bite, narrow maxilla (upper jaw), and underdeveloped upper jaw. This process is disclosed, for example, in U.S. Pat. No. 11,045,283.

MARPE has been used to expand the upper jaw using mid-palatal suture separation, which in general terms involves sideways movement of the two halves of the upper jaw. However, a common side effect of MARPE is upper posterior teeth tilting. Another side effect of MARPE is facial bone structure modification that causes aesthetic issues for the patient due to the implicit maxilla bone movement induced by the procedure.

There is a need to reduce and/or avoid one or more of the above unwanted side effects of MARPE.

### SUMMARY OF THE INVENTION

An exemplary mid-palatal piezocorticomy guide for a maxilla having a palatial region within a palatial perimeter may comprise a retainer for retaining the guide to the maxilla at a point outside the palatial perimeter and a base suspended from the retainer by at least one post extending from the retainer across the palatial perimeter, the base being configured to be proximal to the palatial region of the maxilla. An exemplary mid-palatal piezocorticomy guide for a maxilla having a palatial region within a palatial perimeter may be further configured such that the base has at least one slotted space through its thickness through which a palatial region is accessible when the retainer is coupled to the maxilla.

In addition to or in combination with any other previously described embodiment, an exemplary mid-palatal piezocorticomy guide previously described may further comprise a plurality of slotted spaces through the thickness of the base.

In addition to or in combination with any other previously described embodiment, an exemplary mid-palatal piezocorticomy guide previously described may be configured such that the plurality of slotted spaces are aligned with one another.

In addition to or in combination with any other previously described embodiment, an exemplary mid-palatal piezocorticomy guide previously described may be configured such that a position and length of the plurality of slotted spaces are preselected based on a pre-planned incision procedure.

In addition to or in combination with any other previously described embodiment, an exemplary mid-palatal piezocorticomy guide previously described may be configured such that a position and length of at least two of the plurality of slotted spaces are unequal.

In addition to or in combination with any other previously described embodiment, an exemplary mid-palatal piezocorticomy guide previously described may further comprise a plurality of posts.

In addition to or in combination with any other previously described embodiment, an exemplary mid-palatal piezocorticomy guide previously described may be configured such that the posts have rectangular cross-sections.

In addition to or in combination with any other previously described embodiment, an exemplary mid-palatal piezocorticomy guide previously described may be configured such that the posts are metallic.

Another exemplary mid-palatal piezocorticomy guide for a maxilla having a palatial region within a palatial perimeter may comprise a retainer for retaining the guide to the maxilla at a point outside the palatial perimeter, a base suspended from the retainer by a plurality of posts extending from the retainer across the palatial perimeter, the base being configured to be in contact with a portion of the palatial region of the maxilla. Such an exemplary mid-palatal piezocorticomy guide for a maxilla having a palatial region within a palatial perimeter may be further configured such that the base provides access to select areas of the portion of the palatial region through a plurality of slots through its thickness.

In addition to or in combination with any other previously described embodiment, an exemplary mid-palatal piezocorticomy guide previously described may be configured such that a position and length of at least two of the plurality of slots are unequal.

In addition to or in combination with any other previously described embodiment, an exemplary mid-palatal piezocorticomy guide previously described may further comprise four posts.

Any other previously described embodiment or combinations of embodiments may be incorporated into a method of mini-screw-assisted rapid palatal expansion using the exemplary maxillary incision guide of any previously described embodiment or combinations of embodiments.

### BRIEF DESCRIPTION OF THE DRAWINGS

FIG. 1 illustrates an exemplary maxilla environment.

FIGS. 2A-B illustrate an exemplary embodiment of an incision operation on the maxilla.

FIGS. 3 and 4A-B illustrate an exemplary embodiment of the result of the incision operation on the maxilla conducted in FIGS. 2A-B.

FIGS. 5A-B illustrate an exemplary mid-palatal piezocrticomy guide.

FIGS. 6A-B illustrate an exemplary maxilla after use of an exemplary mid-palatal piezocorticomy guide.

FIGS. 7A-B illustrate an exemplary MARPE affixed to an exemplary maxilla after use of an exemplary mid-palatal piezocorticomy guide.

FIGS. 8A-B illustrate the expansion of an exemplary MARPE affixed to an exemplary maxilla after use of an exemplary mid-palatal piezocorticomy guide.

FIGS. 9A-B illustrate the skeletal results following use of an exemplary MARPE affixed to an exemplary maxilla after use of an exemplary mid-palatal piezocorticomy guide as per FIGS. 6B, 7B, 8B, and 9B.

FIG. 10 illustrates an exemplary methodology of fabricating an exemplary piezocorticomy guide.

In the drawings like characters of reference indicate corresponding parts in the different figures. The drawing figures, elements and other depictions should be understood as being interchangeable and may be combined in any like manner in accordance with the disclosures and objectives recited herein.

### DETAILED DESCRIPTION

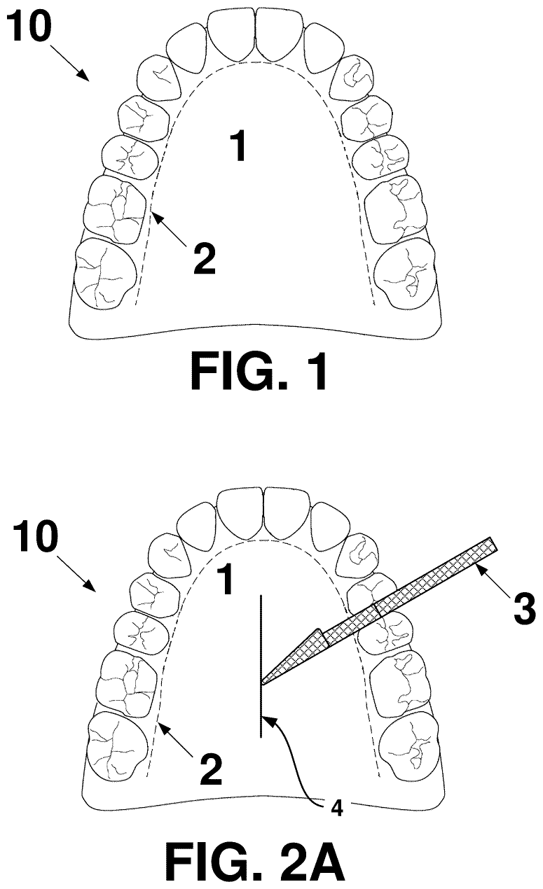

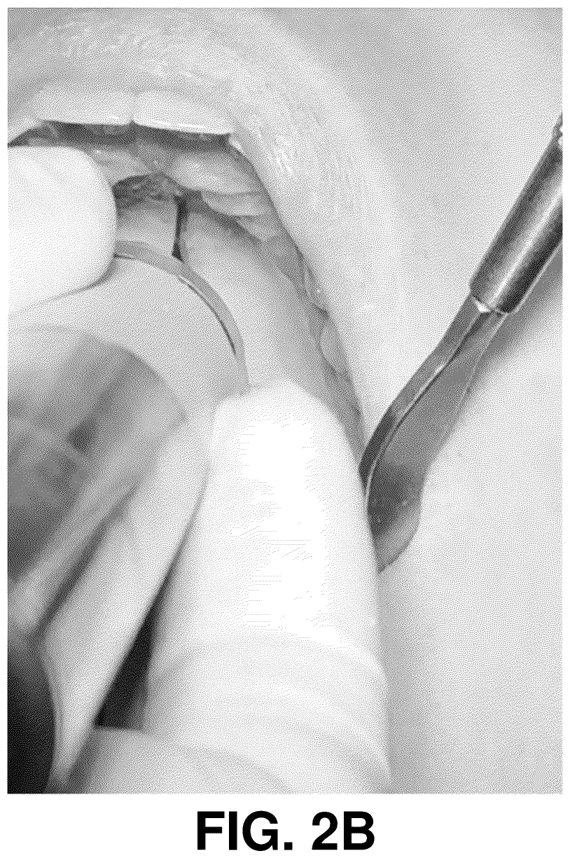

With reference to FIGS. 1 and 2A, an exemplary maxilla 10 may be shown with a palatial area 1 within palatial perimeter 2. As illustrated in FIGS. 2A-B, a prior art method of palatial expansion may include use of an incision device 3, e.g., a scalpel, piezocorticotomy tool, or like cutting tool, being used within the palatial area 1 along a path 4. In an exemplary embodiment, path 4 may be straight, intermittent, or other angles and orientations as deemed necessary by the practitioner. In another exemplary embodiment, path 4 may be within the palatial perimeter 2.

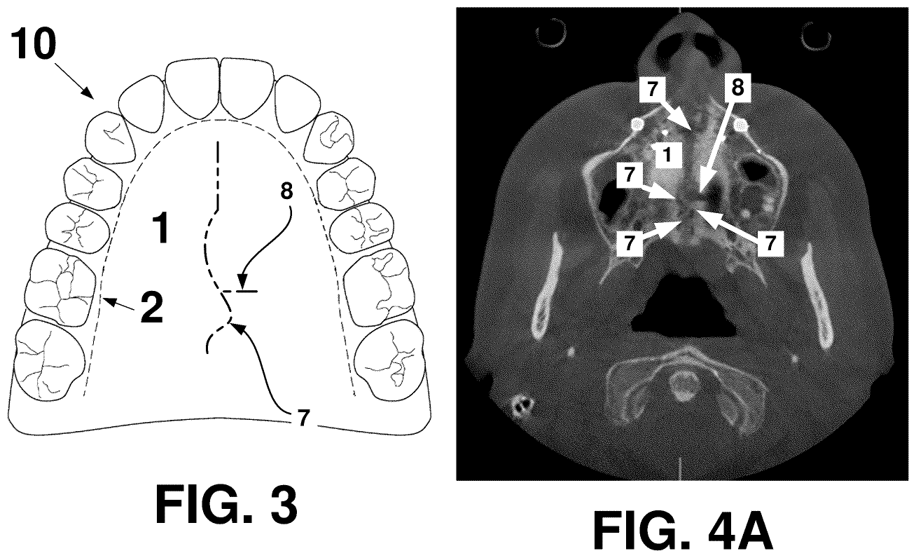

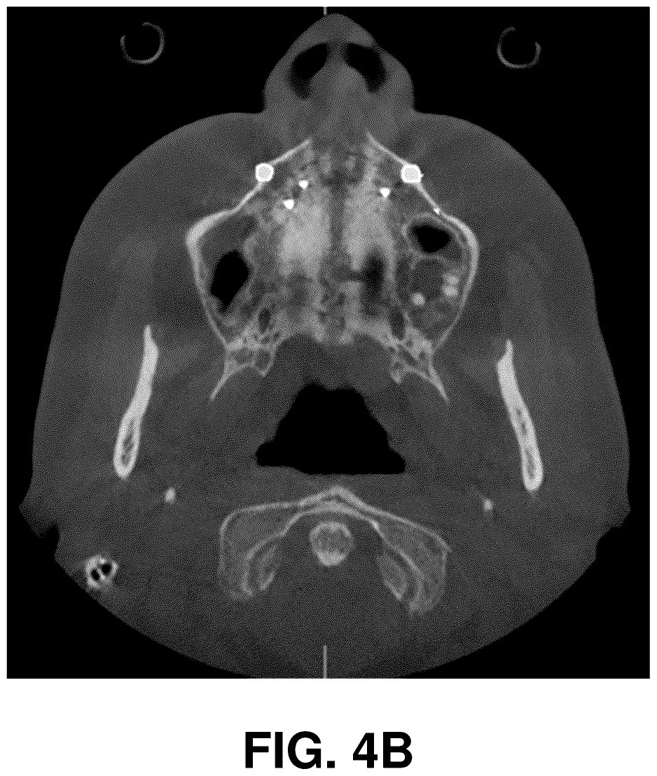

Referring to FIGS. 3 and 4A-B, an exemplary maxilla 10 may be shown with side effects from the procedure illustrated in FIGS. 2A-B. As illustrated in FIG. 3, an exemplary side effect of the procedure of FIGS. 2A-B may be an asymmetrical fracture line 7 and/or transverse suture 8. FIGS. 4A-B illustrate the Cone-beam computed tomography systems (CBCT) scan of the maxilla 10 depicted in FIG. 2B with exemplary fracture lines 7 and/or transverse suture 8. It has been found that the exemplary illustrated and practiced results of FIGS. 3 and 4A-B, respectively, that follow the illustrated procedure in FIG. 2A and the practiced procedure in FIG. 2B, respectively, causes aesthetic issues for the patient in terms of asymmetrical facial appearance following maxillary healing following subsequent use of MARPE. Thus, the issue presented by the current procedures requires improvement to avoid these unwanted results in the predicate steps before implementation of MARPE.

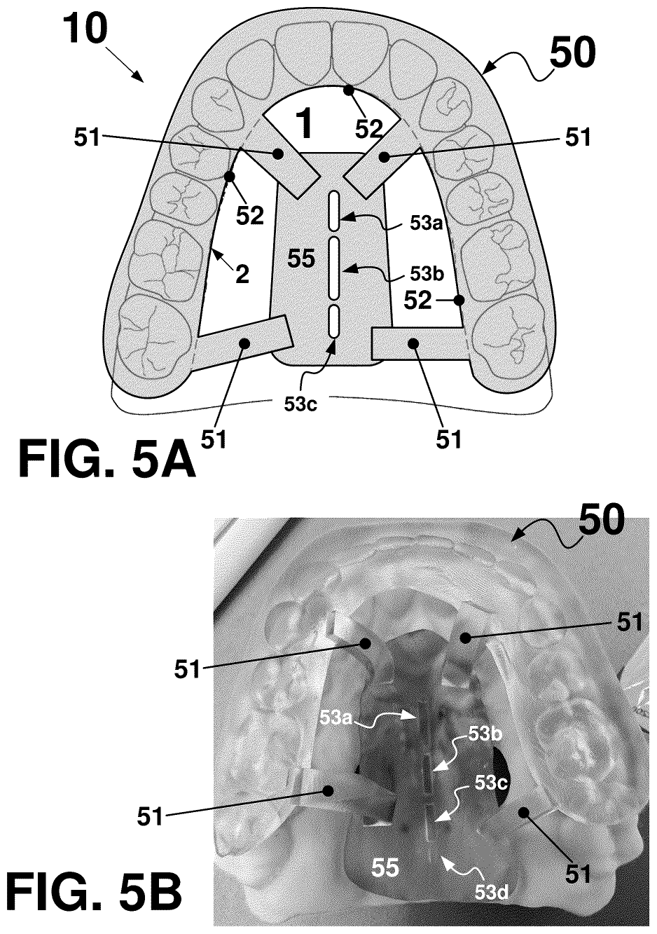

With reference to FIGS. 5A-B, an exemplary piezocorticomy guide 50 may be situated on maxilla 10 so that a retainer 52 anchors about one or more teeth extending from maxilla 10. An exemplary palatal base 55 of the guide 50 may be proximal to palatial region 1, and in an exemplary embodiment, may be in contact with the palatial region 1. Palatal base 55 may be coupled to the retainer 52 of an exemplary guide 50 via posts 51 extending from base 55 to region 52. In a preferred embodiment, posts 51 may be rectangular in cross-section, although other cross-sections may be employed. In another exemplary aspect, exemplary posts 51 may be made of flexible metal so that the posts can be bent into a particular position and retain that position during a procedure.

In an exemplary embodiment, posts 51 may allow guide 50 to be retained outside of the palatial perimeter 2 while also allowing guide 50 to extend into the palatial region 1 for the situation of base 55 therein. An exemplary guide 50 may be made of any known material capable of performing the procedures described. For example, an exemplary guide 50 may be of similar material composition of known maxillary retainers whereby retainer 52 may be a labial metal arch wire while base 55 may be an acrylic or like polymer material such as types used with Hawley retainers. In a preferred embodiment, guide 50 may be a thermoplastic or other polymer-based composite material, such as, for example materials formulated with ethoxylated bisphenol A-dimethacrylate (Bis-EMA), urethane dimethacrylate (UDMA), and triethylene glycol dimethacrylate (TEGDMA). Other exemplary materials may be any known or suitable additive manufacturing resins (i.e., 3D printing resins) for ultraviolet digital light processing (UV-DLP) 3D printers. In a preferred embodiment, exemplary printed material thickness should exceed 2.50 mm for rigidity and the width of the piezocision notches (slots 53a-d) is 1.2-4.0 mm, with 3-4 notches/slots per piezocortical guide. In an alternative embodiment, an exemplary retainer 52 may be formed using known techniques for other dental procedures, such as the types of retainers illustrated and described in Lyros I, Tsolakis I A, Maroulakos M P, Fora E, Lykogeorgos T, Dalampira M, Tsolakis A I. Orthodontic Retainers-A Critical Review. Children (Basel). 2023 Jan. 28;10(2):230. doi: 10.3390/children10020230. PMID: 36832359; PMCID: PMC9954726, the details, figures, and suggestions therein being incorporated herein by reference in their entirety. An exemplary base 55 may be coupled to a pre-formed retainer 55 using pre-fabricated posts 51 that may be vibration welded, molded, or otherwise chemically or mechanically adhered to region 52. In yet another embodiment, an exemplary guide 50 may be fabricated from overlaying a mold on a resin or composite replica of the desired maxilla 10, as may be shown in FIG. 5B. In yet another embodiment, the piezocortical guide 50 may be printed/sintered from a metal alloy or any other resin modification to the same.

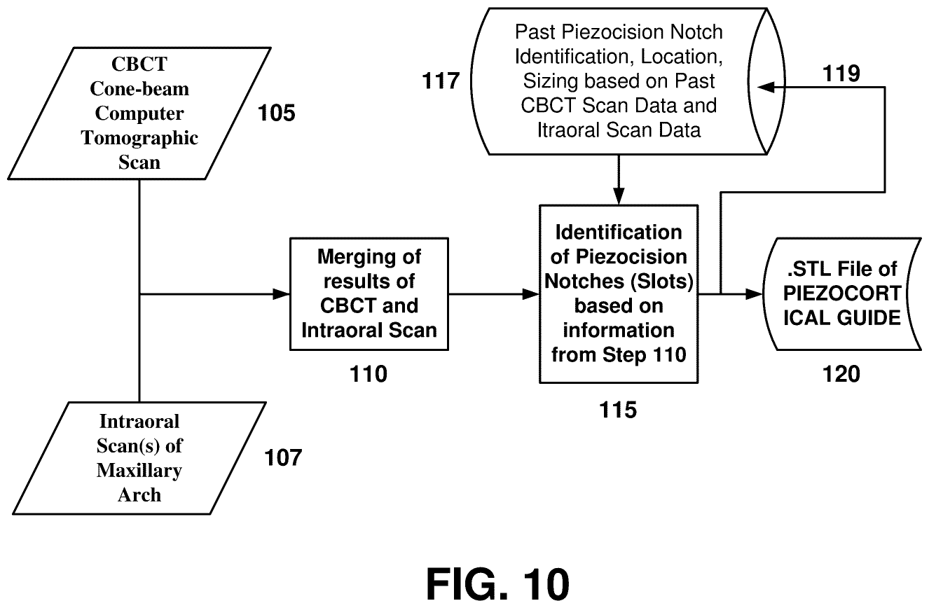

In another exemplary embodiment as illustratively provided in the block method diagram of FIG. 10, 3D printing techniques may allow for single-unit guide 50 being printed with preliminary 3D designing of the piezocortical guide 50 as a “.stl” file. In one aspect, an exemplary “.stl” file of the guide 50 may be transferred to the connected 3D printer to be printed on a platform with similar resin supports and foundations. Further handling of the printed piezocortical guide 50 may depend on the resin manufacturer instructions and recommendations, and may include post-printing cleaning, drying, excess resin elimination, and curing. In another aspect of this exemplary embodiment, slight post-curing polishing of the 3D printed guide 50 may be used to remove printing supports and connectors to the final guide 50 formed thereby. According to this exemplary embodiment of a guide 50 creation methodology, 3D construction and design of the piezocortical guide 50 may include a first step 105 of acquiring Cone-beam computer tonographic (CBCT) scan data of the patient's midfacial region and/or a step 107 of acquiring intraoral scans of the maxillary arch 10 and palatial region 1. An exemplary second step 110 may include an analysis of the Cone-beam computer tonographic (CBCT) scan data of the patient's midfacial region with the intraoral scans of the maxillary arch 10 and palatial region 1 and a merging of the results of the CBCT scan data and itraoral scans using known scanning overlay techniques, such as, for example, the three-dimensional modeling methodologies discussed in Aser M. Eissa, Ibrahim F. Shaker, A. M. Abdel-Wahab, Alaa A L Din I. Awad, Integration of multi-photos and laser scanner data to form a complete 3d model, Ain Shams Engineering Journal, Volume 14, Issue 5, May 2023, the entire contents of which being incorporated herein by reference. In an exemplary third step 115, an exemplary method may include an identification of the location, size, spacing, and other dimensional data of the required piezocision notches/slots 53a-d from the results from step 110.

In an exemplary aspect of an exemplary step 115, a practitioner's prior piezocision identification and specifications or like a priori information of the kind may be stored on an accessible memory-based digital repository 117 for review by an artificial intelligence engine known to those skilled in the art as part of step 115, and, based on the data from step 110, the practitioner may use the prior piezocision data in conjunction with the CBCT scan data and intraoral scans to obtain a piezocision map for in step 115 based on interpolations of the practitioner's past practices and data with the option to manually adjust the results and the final configuration data. In another exemplary aspect, the results of the step 115 may subsequently be stored via storage step 119 in the repository 117 for use in subsequent piezocision identification steps 115. In an exemplary fourth step 120, an exemplary method may produce a “.stl” file of the piezocortical guide 50 for rendering and/or manufacturing from a variety of sources, e.g., 3D Printers and/or CNC machines. Accordingly, this particular methodology may ensure precise locating of the mid-palatal suture, nasal septum, and its relationship to the surrounding anatomical structures. In a preferred embodiment, piezocorticotomy procedure planning and execution may be the direct predictor of timely maxillary skeletal separation, reduced risk of associated peri-maxillary fractures, symmetrical position of the nasal septum post-expansion.

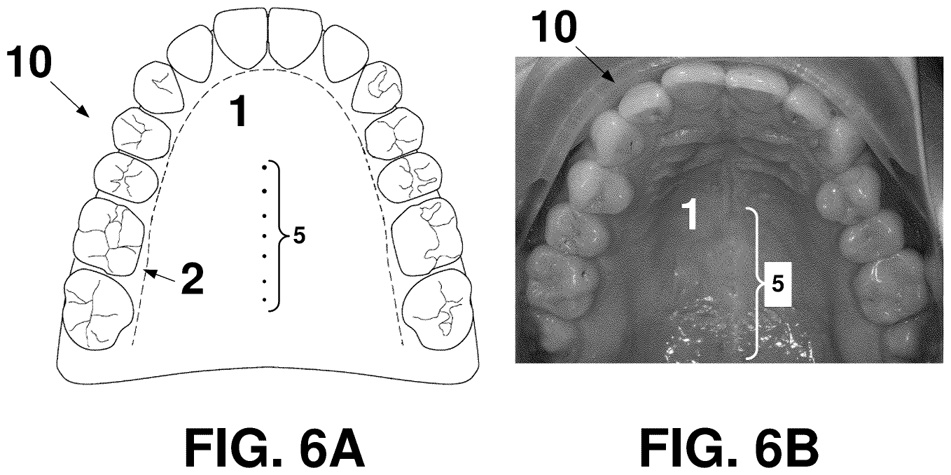

With continued reference to FIGS. 5A-B, an exemplary piezocorticomy guide 50 may have within its base 55 one or more slots 53a, 53b, 53c, and/or 53d. According to the inventive methodologies disclosed, a practitioner may pre-select the size and positions of slots 53a-d for the particular patient. Once selected, the practitioner may perform the incision procedure discussed with reference to FIGS. 2A-B but operate only within slots 53a-d. Thus, an exemplary guide 50 may be used as a form of maxillary operation stencil customized to the patient's unique maxillary conformation. This stencil-type prosthesis provides the practitioner with increased flexibility during the maxillary incision procedure and allows the necessary cutting procedure to take place without the errors that accompany normal incision practice, which is a byproduct of using cutting tools in the mouth of a patient for which there is limited space to properly maneuver the cutting tool vis-à-vis the palatial region 1. FIGS. 6A-B may illustrate the results of the incision techniques described in FIGS. 2A-B with the difference being that such incisions take place via use of base 55 of an exemplary guide 50 installed on the maxilla 10. In comparison to the uneven incision lines 4, 7, and 8, an exemplary guided incision line 5 may be more uniform and without the deviations discussed in and illustrated in FIGS. 3 and 4A-B.

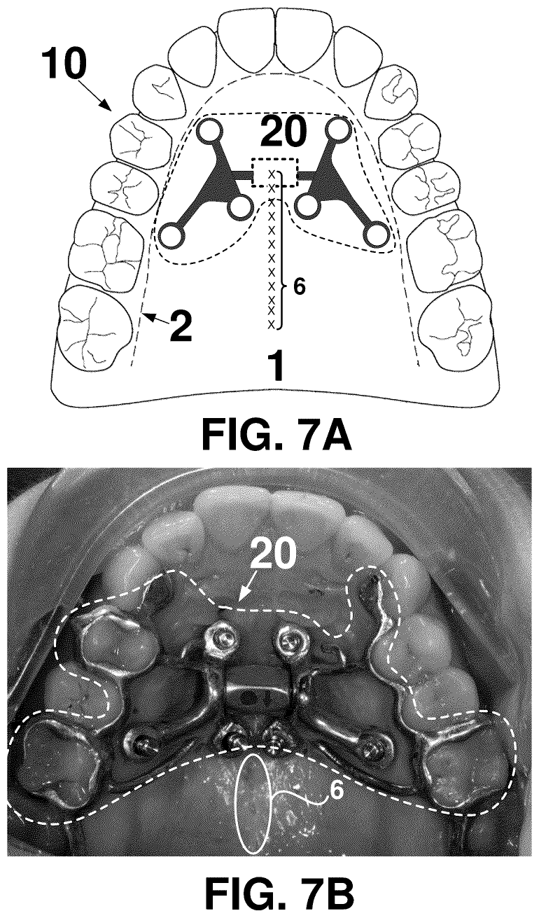

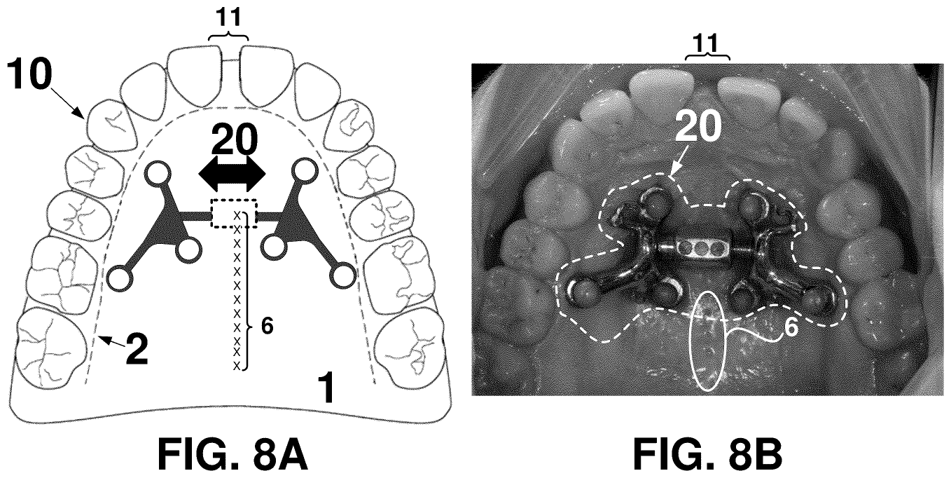

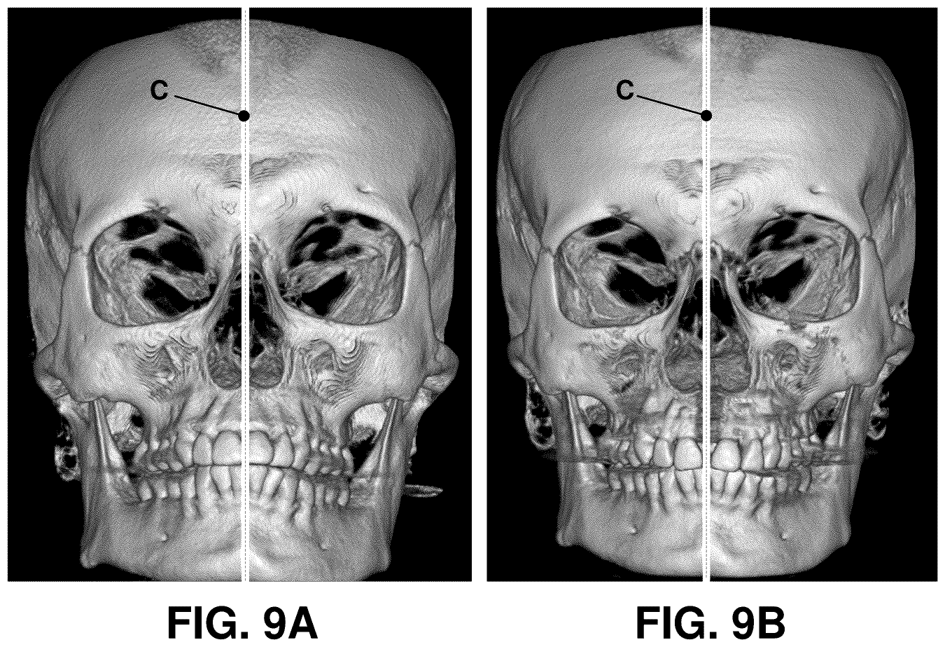

With reference to FIGS. 7A-B, an exemplary maxilla 10 may be shown with healed sutures 6 from a guided incision line 5 during installation of an exemplary MARP device 20. As the exemplary MARP device 20 is expanded as illustratively provided for in FIGS. 8A-B, the maxilla 10 may experience uniform and symmetrical spacing 11 of the maxilla bone structure. A consequence of such uniform expansion may be illustrated by FIGS. 9A-B, where FIG. 9A may illustrate the skeletal structure of the subject prior to corticotomy incision of the maxilla using guide 50, and FIG. 9B may illustrate the skeletal structure of the subject following the incision procedure depicted in FIG. 6B and the MARP procedure depicted in FIGS. 7B and 8B. As may be illustrated in FIGS. 9A-B, an exemplary center line “C” may be substantially in the same location before and after the procedure(s) described with respect to FIGS. 6B, 7B, and 8B.

Clinical results of using piezocorticotomy executed with the pre-planned piezocortical 3D-printed guide 50 may provide:
1) maxillary skeletal separation within 14-21 days when all provider recommendations are followed;
2) reduction in risk of alveolar fractures, transverse suture separation, and palatine bone fractures;
3) midpalatal separation with both ANS and PNS landmarks separated in a parallel fashion;
4) substantially more predictable maxillary expansion within +/−5 mm;
5) basis for forward midfacial translation; and
6) midfacial downward rotation and correction of the excessive gingival display.

One exemplary evident clinical effect of piezocortically assisted midfacial expansion was the increase in the nasal airway volume due to skeletal separation of the midpalatal suture. Using an exemplary 3D planning of the piezocorticotomy guide may increase midpalatal suture separation to produce an even and symmetrical increase in nasal airway passage volume. Subsequently, nasopharyngeal airway volume may be increased due to predictably parallel separation of the PNS landmarks of the palatine bones and lateral movement of the pterygoid processes of the sphenoid bones.

The invention according to any of the foregoing embodiments may be modified to reduce the extensity and location of the piezocortical notches, but without reducing the most posterior notches crossing the transverse suture.

Many further variations and modifications may suggest themselves to those skilled in art upon making reference to above disclosure and foregoing interrelated and interchangeable illustrative embodiments, which are given by way of example only, and are not intended to limit the scope and spirit of the interrelated embodiments of the invention described herein.

1. A maxillary incision guide for a maxilla having a palatial region within a palatial perimeter, comprising:
a retainer for retaining the guide to the maxilla at a point outside the palatial perimeter;
a base suspended from the retainer by at least one post extending from the retainer across the palatial perimeter, the base being configured to be proximal to the palatial region of the maxilla,
wherein the base has at least one slotted space through its thickness through which a palatial region is accessible when the retainer is coupled to the maxilla.

2. The maxillary incision guide of claim 1, further comprising a plurality of slotted spaces through the thickness of the base.

3. The maxillary incision guide of claim 2, wherein the plurality of slotted spaces are aligned with one another.

4. The maxillary incision guide of claim 2, wherein a position and length of the plurality of slotted spaces are preselected based on a pre-planned incision procedure.

5. The maxillary incision guide of claim 2, wherein a position and length of at least two of the plurality of slotted spaces are unequal.

6. The maxillary incision guide of claim 3, further comprising a plurality of posts.

7. The maxillary incision guide of claim 4, further comprising a plurality of posts.

8. The maxillary incision guide of claim 5, further comprising a plurality of posts.

9. The maxillary incision guide of claim 6, wherein the posts have rectangular cross-sections.

10. The maxillary incision guide of claim 7, wherein the posts have rectangular cross-sections.

11. The maxillary incision guide of claim 8, wherein the posts have rectangular cross-sections.

12. The maxillary incision guide of claim 6, wherein the posts are metallic.

13. The maxillary incision guide of claim 7, wherein the posts are metallic.

14. The maxillary incision guide of claim 8, wherein the posts are metallic.

15. A maxillary incision guide for a maxilla having a palatial region within a palatial perimeter, comprising:
a retainer for retaining the guide to the maxilla at a point outside the palatial perimeter;
a base suspended from the retainer by a plurality of posts extending from the retainer across the palatial perimeter, the base being configured to be in contact with a portion of the palatial region of the maxilla,
wherein the base provides access to select areas of the portion of the palatial region through a plurality of slots through its thickness.

16. The maxillary incision guide of claim 15, wherein a position and length of at least two of the plurality of slots are unequal.

17. The maxillary incision guide of claim 16, further comprising four posts.

18. The maxillary incision guide of claim 17, wherein each post is metallic.

19. A method of mini-screw-assisted rapid palatal expansion, comprising the steps of: using the maxillary incision guide of claim 1 in the mouth of a user before installing a mini-screw-assisted rapid palatal expander in the mouth of the user.

20. A method of mini-screw-assisted rapid palatal expansion, comprising the step of using the maxillary incision guide of claim 15 in the mouth of a user before installing a mini-screw-assisted rapid palatal expander in the mouth of the user.
